(19) United States
(12) Patent Application Publication
(10) Pub. No.: US 2026/0096795 A1
(43) Pub. Date: Apr. 9, 2026

(54) CEREBRAL EMBOLIC PROTECTION SELECTION METHOD FOR TAVI PROCEDURES

(71) Applicant: KONINKLIJKE PHILIPS N.V., EINDHOVEN (NL)

(72) Inventors: HOLGER SCHMITT, LUETJENSEE (DE); MICHAEL GRASS, BUCHHOLZ IN DER NORDHEIDE (DE); CHRISTIAN HAASE, HAMBURG (DE); ARJEN VAN DER HORST, TILBURG (NL); HANNES NICKISCH, HAMBURG (DE); MANINDRANATH VEMBAR, TWINSBURG, OH (US)

(21) Appl. No.: 19/112,890

(22) PCT Filed: Sep. 11, 2023
(86) PCT No.: PCT/EP2023/074827

§ 371 Date: Mar. 18, 2025

Related U.S. Application Data
(60) Provisional application No. 63/408,134, filed on Sep. 20, 2022.

(51) Int. Cl. A61B 6/50; A61B 6/00; A61F 2/24; G06T 7/00; G06V 10/26; G06V 10/82; G16H 20/40

(52) U.S. Cl. CPC A61B 6/504; A61B 6/507; A61B 6/5217; A61F 2/2427; G06T 7/0012; G06V 10/26; G06V 10/82; G16H 20/40; G06T 2207/10081; G06T 2207/20081; G06T 2207/20084; G06T 2207/30104; G06V 2201/03

(57) ABSTRACT

Whether to deploy a cerebral embolic protection (CEP) device is determined by initially retrieving one or more image of at least part of an aorta. The image includes an aortic valve. The image is segmented to identify the aortic valve, an aortic arch, and a plurality of branching blood vessels downstream of the aortic valve. Plaque in a segment of the image at or adjacent the aortic valve is identified, and a vulnerability score associated with the plaque is generated. Dynamics of blood flow in the aortic arch and at least one of the plurality of branching blood vessels is evaluated. It is then determined whether a CEP device should be deployed at least partially based on the vulnerability score. A CEP device is selected at least partially based on the dynamics of blood flow in the aortic arch.

### FIELD OF THE INVENTION

The present disclosure generally relates to systems and methods for determining the necessity for cerebral embolic protection (CEP) during a procedure, such as a surgery, and for selecting appropriate such protections. In particular, the disclosure relates to determining appropriate CEP devices for use during TAVI or TAVR procedures.

### BACKGROUND

Medical procedures involving arterial vessels often create a risk that plaque will detach from walls of the blood vessel. For example, a transcatheter aortic valve implementation or replacement (TAVI/TAVR used interchangeably) may create a risk of plaque debris detaching and traveling with the blood through various arteries, potentially resulting in a stroke, among other potential repercussions.

Cerebral embolic protection (CEP) devices have been available for some time and take several forms. Two examples are the Sentinel system by Boston Scientific, and the TriGuard by Keystone Heart. The purpose of these devices is to capture plaque debris which gets detached during TAVI procedures while inserting the artificial valve. Accordingly, the CEP devices typically function as filters that filter blood flowing into particular vessels branching off of the aortic arch. Typically, there are three major arteries extending from the aortic arch that might require protection. While the Sentinel covers only the innominate and left common carotid artery, the TriGuard in addition covers the left subclavian artery from which the left vertebral artery arises, thereby covering three major arteries extending from the aortic arch.

Not all patients require or even benefit from the use of a CEP device in the context of procedures such as TAVI. Further, the use of a CEP device may increase time and/or cost associated with a procedure, and may not be desirable in all cases. Therefore, there is a need to determine in advance of a TAVI or TAVR procedure whether a patient will benefit from the use of a CEP device. Further, as noted above, while there are three major cerebral vessels branching off the aortic arch, not all CEP devices cover all three, and if a CEP device is determined to be appropriate, not all patients will benefit from covering all vessels.

As such, there is a need for a pre-operative method for identifying patients who may benefit from CEP devices during TAVI procedures, as well as a method for selecting and implanting an appropriate CEP device once such a device is determined to be appropriate.

### SUMMARY

Methods and systems are provided for selecting and implementing cerebral embolic protection devices in the context of a procedure, such as a TAVI surgery.

The method assumes that patients will benefit from a CEP device during a TAVI procedure if they have plaque in the area of the aorta where the new valve is deployed. Further, a device covering all three cerebral branches of the aorta will provide a benefit if the angle between aorta and left subclavian artery is relatively small. The presence of both features can be detected in computed tomography (CT) images, such as spectral CT images.

In some embodiments, a method is provided for deploying a cerebral embolic protection (CEP) device, where the method includes retrieving one or more image of at least part of an aorta. The one or more image includes an aortic valve. The method includes segmenting the one or more image to identify the aortic valve, an aortic arch, and a plurality of branching blood vessels downstream of the aortic valve.

The method then identifies plaque in a segment of the one or more image at or adjacent the aortic valve and generates a vulnerability score associated with the identified plaque. The method then evaluates dynamics of blood flow in the aortic arch and at least one of the plurality of branching blood vessels.

The method then determines that a CEP device should be deployed at least partially based on the vulnerability score and selects a CEP device from a plurality of potential CEP devices at least partially based on the dynamics of blood flow in the aortic arch.

In some embodiments, the vulnerability score is correlated with a risk that at least some of the plaque is detached during a surgical procedure applied to the aortic valve. Such a procedure may be a transcatheter aortic valve implantation (TAVI) procedure applied to the aortic valve.

In some embodiments, the vulnerability score is based at least partially on the type of implant to be used in the TAVI procedure.

In some embodiments, the vulnerability score is based at least partially on a total plaque volume and a spatial configuration of the plaque at or adjacent the aortic valve and a fraction of lipid in the total plaque volume. In some such embodiments, the vulnerability score is further based on morphological factors associated with the plaque.

In some embodiments, the vulnerability score is determined by an artificial intelligence (AI) based model trained on known outcomes of previous surgical interventions correlated with corresponding historical images of aortas, where the historical images include a corresponding aortic valve.

In some embodiments, the determination that a CEP device should be deployed is based at least partially on the vulnerability score and not the branching angle, and wherein the CEP device to be deployed is selected based at least partially on the branching angle and not the vulnerability score.

In some embodiments, the one or more image is one or more computed tomography (CT) image, and the segmenting of the one or more image is implemented by an artificial intelligence (AI) based model to identify the aortic arch and a left subclavian artery (LSA).

In some embodiments, the method includes identifying a branching angle between the LSA and the aortic arch, and the selection of the CEP device is based on a fluid dynamics model of blood flow between the aortic arch and the LSA, the fluid dynamics model determining a likelihood that plaque in the aortic arch will enter the LSA based at least partially on the identified branching angle.

In some embodiments, the fluid dynamics model is further based on a size of the LSA, and the size of the LSA is determined from the image or is independently known.

In some embodiments, the plurality of branching blood vessels include the brachiocephalic artery, the left common carotid artery (CCA) and the LSA. A first CEP device of the plurality of CEP devices covers the brachiocephalic artery and the CCA, but not the LSA and a second CEP device of the plurality of CEP devices covers the brachiocephalic artery, the CCA, and the LSA.

In some embodiments, the branching angle is between a left subclavian artery (LSA) and the aortic arch. The method then includes determining whether a size of the LSA is larger than a threshold size, and a first CEP device of the plurality of CEP devices covers the brachiocephalic artery and the CCA but not the LSA, and where a second CEP device of the plurality of CEP devices covers the brachiocephalic artery, the CCA, and the LSA. Once the method determines that a CEP should be deployed and that either the LSA is larger than the threshold size or the branching angle is larger than a threshold angle, the method further determines that the second CEP device should be deployed.

Also provided herein is a system for deploying a CEP device. The system includes a plurality of implantable potential CEP devices, a memory for storing a plurality of instructions, and processor circuitry that couples with the memory and is configured to execute instructions to implement the method described above.

The instructions are executed in order to retrieve one or more image of at least part of an aorta to be processed, the one or more image including an aortic valve, segment the one or more image to identify the aortic valve, an aortic arch, and a plurality of branching blood vessels downstream of the aortic valve, identify plaque in a segment of the one or more image at or adjacent the aortic valve, generate a vulnerability score associated with the plaque identified, the vulnerability score being correlated with a risk that at least some of the plaque is detached during a surgical procedure applied to the aortic valve, determine that a CEP device should be deployed based on the vulnerability score, identify a branching angle between one of the plurality of branching blood vessels and the aortic arch, and select a CEP device from a plurality of the plurality of implantable potential CEP devices at least partially based on the branching angle.

The selected CEP device is then implanted prior to performing the surgical procedure.

In some embodiments, the system includes a computed tomography (CT) imaging device. The processor circuitry may then retrieve the one or more image from the imaging device.

### BRIEF DESCRIPTION OF THE DRAWINGS

FIG. 1 is a schematic diagram of a system according to one embodiment of the present disclosure.

FIG. 2 illustrates a heart and aorta to be evaluated by a method in accordance with the present disclosure.

FIG. 3A illustrates an aortic arch evaluated by a method in accordance with the present disclosure.

FIG. 3B shows a scan of an aortic valve evaluated by a method in accordance with the present disclosure.

FIG. 3C shows a scan of an aortic arch evaluated by a method in accordance with the present disclosure.

FIG. 4 illustrates a first potential CEP device deployed in accordance with a method of this disclosure.

FIG. 5 illustrates a second potential CEP device deployed in accordance with a method of this disclosure.

FIG. 6 illustrates a method for processing images in accordance with this disclosure.

### DETAILED DESCRIPTION OF THE PREFERRED EMBODIMENTS

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

It is important to note that the embodiments disclosed are only examples of the many advantageous uses of the innovative teachings herein. In general, statements made in the specification of the present application do not necessarily limit any of the various claimed disclosures. Moreover, some statements may apply to some inventive features but not to others. In general, unless otherwise indicated, singular elements may be in plural and vice versa with no loss of generality.

A pre-operative method is described for identifying patients who may benefit from CEP devices during TAVI procedures. Typically, if a patient is scheduled for a TAVI procedure, the method described herein may be applied to determine if the patient is a good candidate for a CEP device. Once a patient is determined to be a good candidate for such a CEP device, the method then involves selecting and, in some embodiments, implanting an appropriate CEP device in advance of the TAVI procedure.

The method assumes that patients will benefit from a CEP device during a TAVI procedure if they have plaque in the area of the aorta where the new valve is deployed, and if the composition of such plaque implies that it is likely to detach. Further, a device covering all three cerebral branches of the aorta will provide a benefit if plaque in the blood flow leaving the operation location is likely to flow into the left subclavian artery. To evaluate such a likelihood, the method may employ a fluid dynamics based analysis, or a proxy for such an analysis. For example, such a risk may be less likely if the angle between the aortic arch and left subclavian artery (LSA) is relatively small. Similarly, a smaller LSA implies less risk that plaque will enter the vessel. The presence of such anatomical features can be detected in medical images obtained from the patient.

As such, the method typically involves retrieving an image of at least part of an aorta in which the TAVI procedure is to be performed. Such an image is typically a CT image and may be a spectral CT image and captures the aortic valve, the left ventricular outflow tract, and the aortic arch of the patient. In some embodiments, a CT unit may be employed to generate one or more images as part of the method. However, in many embodiments, imaging would have been performed prior to surgery, and such existing imaging may be usable to implement the method described herein.

Further, in some embodiment, pre-surgical imaging may take a form other than CT. In medical imaging other than CT, such as Magnetic Resonance Imaging (MRI) or Positron Emission Tomography (PET), different methods may be used for processing images, and resulting images may take different forms. In this disclosure, embodiments are discussed in terms of CT imaging. However, it will be understood that the methods and systems described herein may be used in the context of other imaging modalities as well.

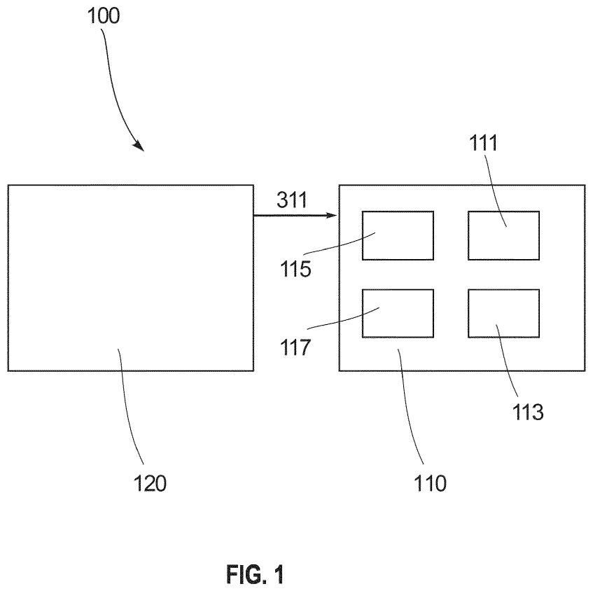

FIG. 1 is a schematic diagram of a system 100 according to one embodiment of the present disclosure. As shown, the system 100 typically includes a processing device 110 and an imaging device 120.

The processing device 110 may apply processing routines to images or measured data, such as projection data, received from the image device 120. The processing device 110 may include a memory 113 and processor circuitry 111. The memory 113 may store a plurality of instructions. The processor circuitry 111 may couple to the memory 113 and may be configured to execute the instructions. The instructions stored in the memory 113 may comprise processing routines, as well as data associated with processing routines, such as machine learning algorithms, and various filters for processing images. While all data is described as being stored in the memory 113, it will be understood that in some embodiments, some data may be stored in a database, which may itself either be stored in the memory or stored in a discrete remote system.

The processing device 110 may further include an input 115 and an output 117. The input 115 may receive information, such as images or measured data, from the imaging device 120. The output 117 may output information, such as processed images, to a user or a user interface device. The output 117 similarly may output determinations generated by the method described below, such as recommendations. The output may include a monitor or display.

In some embodiments, the processing device 110 may relate to the imaging device 120 directly. In alternate embodiments, the processing device 110 may be distinct from the imaging device 120, such that it receives images or measured data for processing by way of a network or other interface at the input 115.

In some embodiments, the imaging device 120 may include an image data processing device, and a spectral or conventional CT scanning unit for generating the CT projection data when scanning an object (e.g., a patient).

While a system is shown including an imaging device 120 and a processing device 110, it will be understood that the method may be implemented directly on a processing device, as in the context of an image received by way of a network at the input 115. The methods described herein involve processing an image as a component of deploying a cerebral embolic protection (CEP) device, generally in the context of a procedure, such as a TAVI surgery. As noted above, prior to such a procedure, imaging is performed. As such, previously generated imaging may be retrieved by way of the input 115 and evaluated prior to or in place of obtaining a new image.

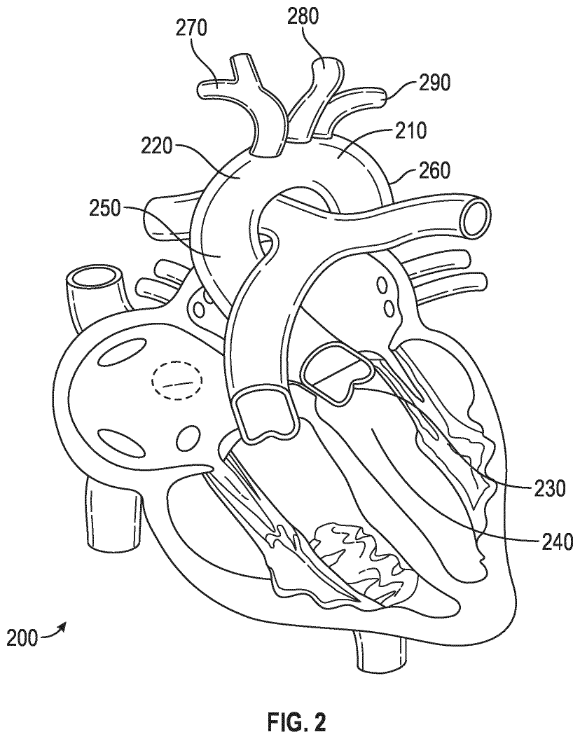

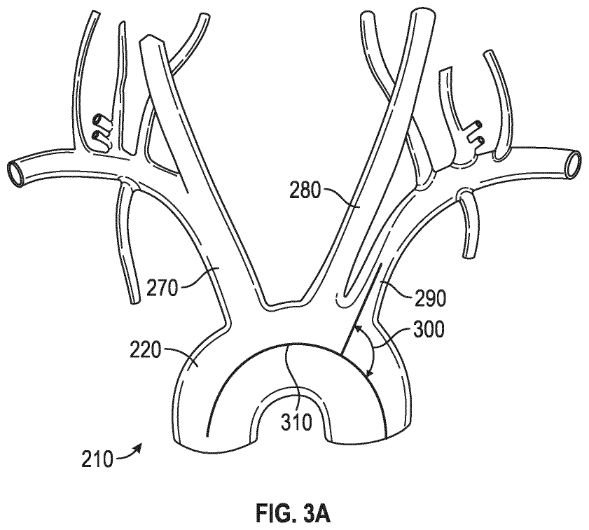

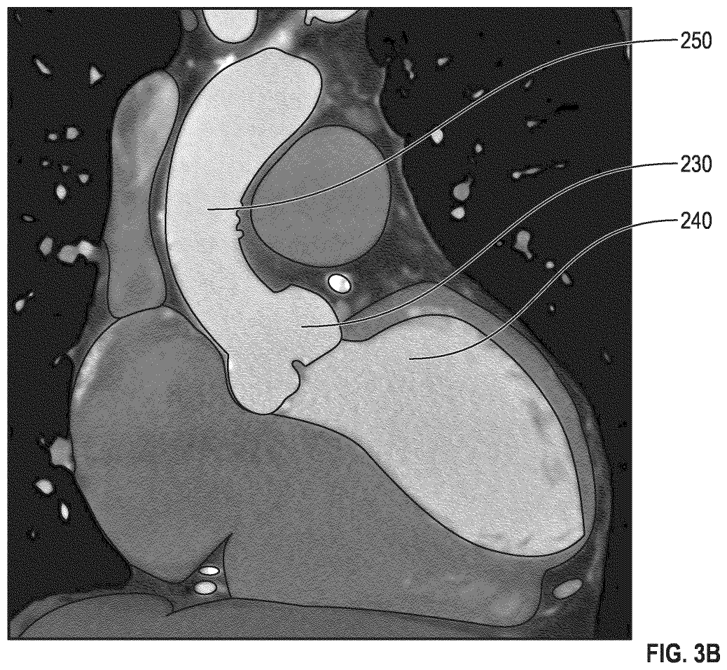

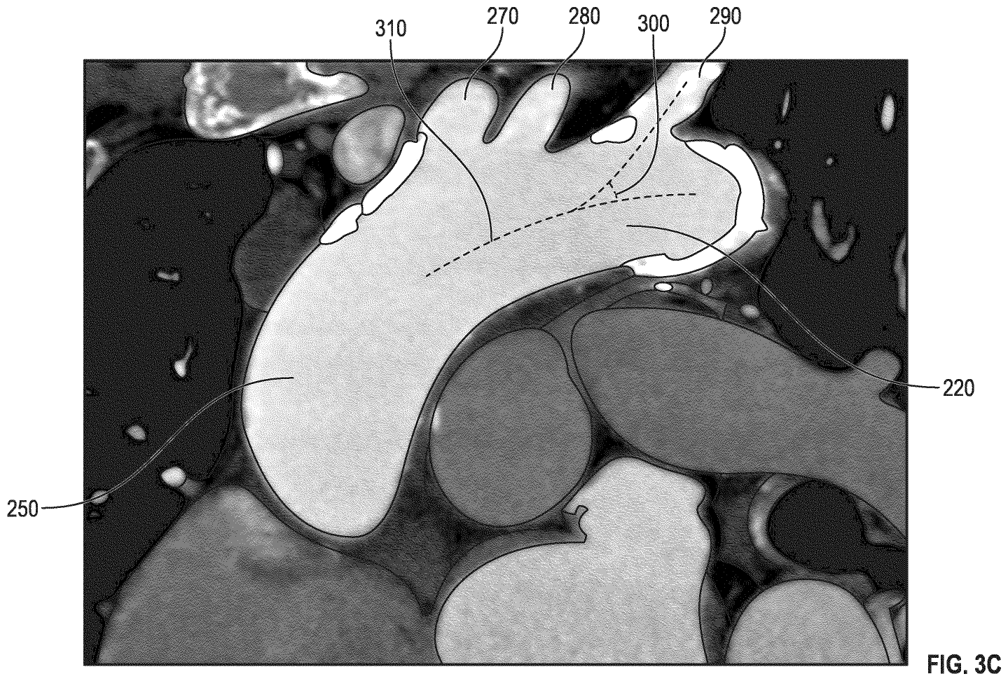

FIG. 2 illustrates a heart 200 and aorta 210 to be evaluated by a method in accordance with the present disclosure. FIG. 3A illustrates a more detailed view of an aortic arch 220 shown in FIG. 2 and evaluated by a method in accordance with the present disclosure.

FIG. 3B shows a scan of an aortic valve 230 evaluated by a method in accordance with the present disclosure. FIG. 3C shows a scan of an aortic arch 220 evaluated by a method in accordance with the present disclosure.

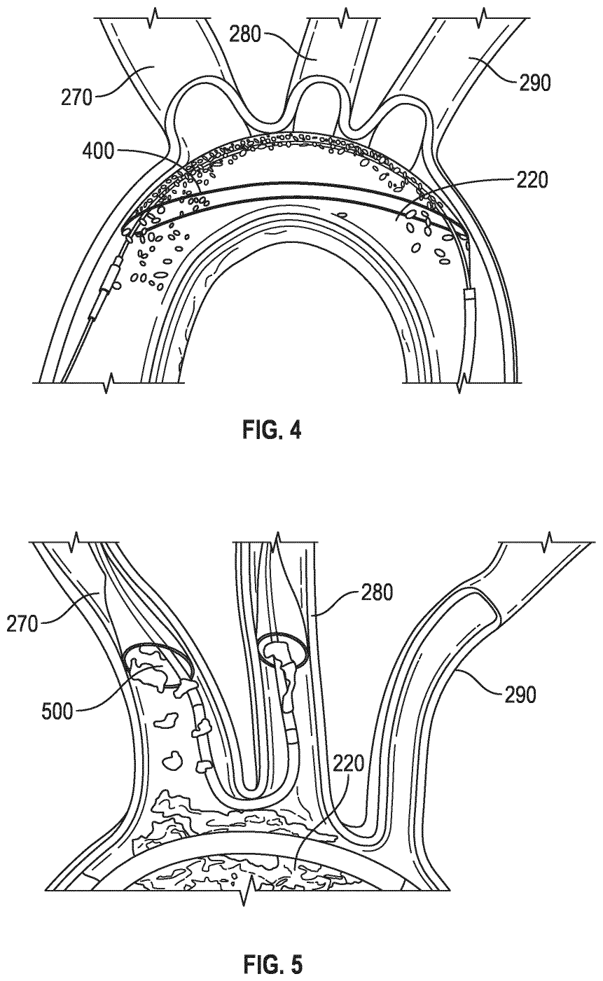

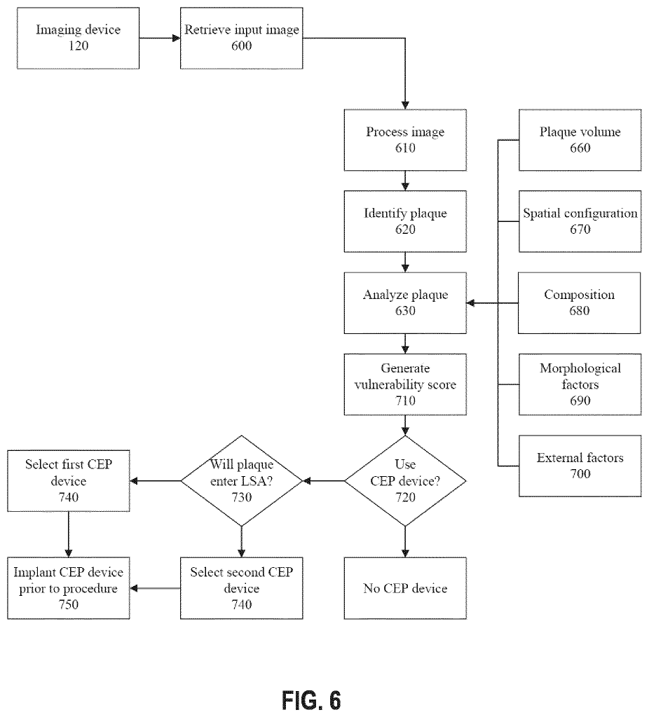

FIG. 4 illustrates a first potential CEP device 400 deployed in accordance with a method of this disclosure. FIG. 5 illustrates a second potential CEP device 500 deployed in accordance with a method of this disclosure. FIG. 6 illustrates a method for processing images in accordance with this disclosure.

As shown in FIGS. 3B and 3C, one or more image of a portion of the patient may be retrieved to evaluate components highlighted in the illustration of FIG. 2. Such images may include the left-ventricular outflow tract (LVOT) 240, aortic valve 230, ascending aorta 250, aortic arch 220, and descending aorta 260. From the aortic arch, three main branching vessels can be identified, including the brachiocephalic artery (also referred to as the brachiocephalic trunk or innominate artery) 270, the left common carotid artery (CCA) 280, and the left subclavian artery (LSA) 290.

As shown in FIGS. 4 and 5, certain CEP devices 400 cover all three main branching vessels 270, 280, 290, while other CEP devices 500 cover only two such vessels, typically the brachiocephalic artery 270 and the CCA 280.

In implementing the method, the system 100 described may first retrieve (600), at an input 115, at least one image of at least part of an aorta 210, such as the aortic arch 220 shown in FIG. 3A. It will be understood that the image or images provided should show the aortic valve 230 and the main branching vessels 270, 280, 290. However, these features are typically in different planes. As such, as shown in FIGS. 3B and 3C, a first image 3B may be provided to show the LVOT 240, the aortic valve 230, and the ascending aorta 250 and a second image 3C may be provided to show the three main branching vessels 270, 280, 290.

It will therefore be understood that in the context of this disclosure, when referring to an image retrieved by the system 100, this may refer to one or more images.

The method then implements various image processing methods (610) and in doing so, identifies plaque (620) in a segment of the image at or adjacent the aortic valve 230 and the left ventricular outflow tract 240, including the ascending aorta 250. The retrieved image (at 600) may be a CT scan of the patient, and in some embodiments, it may be a spectral CT scan.

Accordingly, prior to identifying plaque (at 620), the method may first implement various processing methods (at 610) and apply them to the retrieved image. Such processing methods may include model-based segmentation, which may fit a surface model or voxel masks to the image. Other segmentation approaches may be used as well. Such processing may then be used to identify and segment the various vessels associated with the aorta, including the aortic arch 220 and the three main branching vessels 270, 280, 290.

The segmentation, or other image processing, implemented by the method (at 610) may be by way of an AI based model, such as a convolutional neural network (CNN), and such a method may then be used to identify the aortic arch 220 and the LSA 290, and a branching angle 300 may then be defined between the aortic arch and the LSA.

Once plaque is identified (at 620) and determined to be at or adjacent the aortic valve 230, such plaque may be analyzed (630). Because the TAVI procedure involves implementing or replacing the aortic valve 230, any plaque in the region may be disturbed during the procedure, and as such, the analysis (at 630) determines whether such plaque is likely to become detached during the procedure.

The analysis (at 630) may consider various factors associated with the plaque, including a total volume of plaque 660, a precise spatial configuration of the plaque 670, a specific composition of the plaque 680, such as a fraction of lipid in the total plaque volume, and morphological factors associated with the plaque 690, such as roughness of the plaque surface. The aspects of spatial configuration of the plaque 670 considered may include circumferential coverage as well as coverage of the area where the implant will be deployed.

The analysis (at 630) may also consider external factors 700, such as the type of implant to be used and the precise nature of the surgery to be implemented as well as risk factors associated with the patient. Certain characteristics of the plaque may be more easily identified during the analysis if the image retrieved (at 600) is a spectral CT scan.

The analysis (630) may then generate a vulnerability score (710) associated with the plaque identified (at 620). Such vulnerability score may be a proxy for, and therefore may be correlated with, a risk plaque becoming detached during a surgical procedure applied to the aortic valve. The analysis (630) may be an AI model independent of the segmentation model implemented above (at 610). The AI model may be, for example, a convolutional neural network and may be trained based on historic medical images of a type similar to that retrieved (at 600), such as spectral CT scans, paired with known outcome data with respect to intra- and post-procedural complications. In some embodiments, the AI model may be trained based on the amount of plaque in the area of the procedure prior to and following the procedure, which may indicate the detaching of plaque during the procedure.

In some embodiments, the vulnerability score is based at least partially on the type of implant used in a particular TAVI procedure. In some embodiments, the vulnerability score is based at least partially on total plaque volume and a spatial configuration of the plaque at or adjacent the aortic valve, as well as a fraction of lipid in the total plaque volume. In some embodiments, the vulnerability score is further based on morphological factors associated with the plaque, such as surface roughness.

Once the analysis (at 630) generates the vulnerability score (710), such a score is used to determine (720) whether a CEP device should be deployed for the patient prior to the TAVI procedure. In some embodiments, the vulnerability score (at 710) may be a single value, and the determination (at 720) is simply a determination if the value generated is above or below a threshold. In other embodiments, the vulnerability score (at 710) may include additional factors, including, for example, other risk factors associated with the patient 700, and the determination (at 720) may then collate all risk factors into generating a recommendation.

In some embodiments, further analysis of the image is only performed if the method first determines (at 720) that a CEP device should be deployed. In other embodiments, all image analysis is performed in parallel.

Accordingly, the method further utilizes the segmentation of the aortic arch 220 (performed at 610) to determine (730) whether plaque detached from a wall of the aorta 210 at or adjacent the valve 230 is likely to flow into particular branching blood vessels. While all blood vessels may be evaluated, the LSA 290 in particular is typically evaluated. CEP devices 400, 500 considered for implantation typically cover, and thereby protect, at least the brachiocephalic artery 270 and the left CCA 280. Some CEP devices 400 also cover, and thereby protect, the LSA 290.

Accordingly, the determination (at 730) generally focuses on the likelihood of plaque in blood flowing through the aortic arch 220 entering the LSA 290. This determination (at 730) may be based on a fluid dynamics model and may include an evaluation of a branching angle 300 of at least one of the branching blood vessels relative to a center line 310 of the aortic arch 220. In particular, the determination (at 730) may calculate a branching angle 300 of the LSA 290 relative the aortic arch 220. The calculation may be based on the angle between the local centerline 310 directions in the aortic arch 220 and the proximal LSA 290. A smaller angle 300 between the aortic arch 220 and the LSA 290 indicates that the two vessels are more similar in flow direction and may therefore result in a higher likelihood of plaque debris being washed into the LSA by blood flow in the aorta.

As such, if the angle 300 is smaller, the method may recommend a CEP device 400 that covers all three vessels 270, 280, 290. Alternatively, if the angle 300 is larger, the method may instead recommend a CEP device 500 that covers only the brachiocephalic artery 270 and the left CCA 280. In some embodiments, a threshold angle is used, and therefore defines a risk range within which a CEP device 400 that covers the LSA 290 is recommended. For example, an angle 300 of zero degrees would indicate that the LSA 290 is straight ahead relative to the flow in the aortic arch 220, and an angle of less than 90 degrees would indicate a risk, while an angle greater than 90 degrees would not indicate such a risk.

The method may further consider additional factors as part of a fluid dynamics model to support the determination (730). As such, the segmentation model may allow the method to determine a size of the LSA 290, such as a diameter of the blood vessel. In some embodiments, the size of the LSA 290 is not determined from the segmentation model, but is instead known directly. A larger LSA 290 would result in a higher likelihood that plaque debris is washed into the LSA by blood flow in the aorta 210, and that the method should thereby result in a selection of a CEP device 400 that covers all three vessels. Alternatively, if the LSA 290 is smaller, the method may instead recommend a CEP device 500 that covers only the brachiocephalic artery 270 and the left CCA 280. In some embodiments, the evaluation of the size of the LSA 290 is based on a fraction of the total lumen area of all 3 arteries, and the LSA 290 is then evaluated to determine if it comprises more than one third of the total lumen area. In such an embodiment, if the LSA 290 comprises more than one third of the total lumen area of the three vessels 270, 280, 290, then it would indicate risk and the determination (at 730) would result in a recommendation that a CEP device 400 covering the LSA 290 be used. In other embodiments, the LSA 290 may be evaluated in comparison to the total lumen area of the aortic arch 220.

Accordingly, in some embodiments, the method may rely solely or primarily on the branching angle 300 and may then compare the branching angle to a threshold value, such as ninety degrees, to determine if the LSA 290 should be covered by a CEP device 400. Similarly, the method may rely solely or primarily on the size of the LSA 290, and in such embodiments, the size may be compared to a threshold value, such as one third of the total lumen area of the three vessels 270, 280, 290, to determine if the LSA 290 should be covered. In some embodiments, a more sophisticated fluid dynamics model may be utilized and the determination (at 730) may be based on the branching angle 300, the size of the LSA 290, and in some cases, additional factors, such as rate of blood flow in the patient.

In some such embodiments, an AI model may be utilized to make the determination (at 730). AI models may then be trained based on anatomy and geometry classification, plaque properties, and/or devices to be used, and may be provided with historic imaging, such as spectral CT imaging, paired with known outcome data with respect to complications. The output of such an AI model may be a recommendation on which CEP device should be used. Similar training may be adapted to new devices that enter the market.

In some embodiments, the determination that the CEP device should be deployed (at 720) is based at least partially on the vulnerability score (at 710) and not the branching angle 300, and the CEP device 400, 500 to be deployed is selected based at least partially on the branching angle and not based on the vulnerability score. As such, the determinations may be discrete. Alternatively, the determinations may be used to inform each other.

Once the determination is made (at 730), and assuming the method has also determined that the use of a CEP device is advisable (at 720), the method may select (at 740) a CEP device from a plurality of potential CEP devices at least partially based on the vulnerability score and the branching angle.

### Once Selected, (at 740), the Cep Device May Be Implanted (750) in Advance of a medical procedure, such as TAVI.

In some embodiments, the method is implemented by the system described above 100, either with or without the imaging device 120, which may be a spectral CT device. The system 100 further includes a plurality of implantable potential CEP devices 400, 500, where one of the CEP devices 500 is configured to cover, and thereby protect, only the brachiocephalic artery 270 and the left CCA 280. A second CEP device 400 is instead configured to cover and thereby protect the brachiocephalic artery 270, the left CCA 280, and the LSA 290.

The method is then implemented by the system 100, and upon determining by the system that a CEP device 400, 500 should be deployed based on a vulnerability score, the system further selects a CEP device for implantation. The selected CEP device is then implanted prior to performing the surgical procedure.

While the method and system are described in terms of TAVI/TAVR procedures, similar methods may be applied in adapted forms to identify risk during other vascular interventions. For example, risk may be identified similarly for abdominal artery stenting or carotid artery stenting.

The methods according to the present disclosure may be implemented on a computer as a computer implemented method, or in dedicated hardware, or in a combination of both. Executable code for a method according to the present disclosure may be stored on a computer program product. Examples of computer program products include memory devices, optical storage devices, integrated circuits, servers, online software, etc. Preferably, the computer program product may include non-transitory program code stored on a computer readable medium for performing a method according to the present disclosure when said program product is executed on a computer. In an embodiment, the computer program may include computer program code adapted to perform all the steps of a method according to the present disclosure when the computer program is run on a computer. The computer program may be embodied on a computer readable medium.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

1. A method for deploying a cerebral embolic protection (CEP) device, the method comprising:
retrieving one or more image of at least part of an aorta, the one or more image including an aortic valve;
segmenting the one or more image to identify the aortic valve, an aortic arch, and a plurality of branching blood vessels downstream of the aortic valve;
identifying plaque in a segment of the one or more image at or adjacent the aortic valve;
generating a vulnerability score associated with the identified plaque;
evaluating dynamics of blood flow in the aortic arch and at least one of the plurality of branching blood vessels;
determining that the CEP device should be deployed based on the vulnerability score; and
selecting a CEP device from a plurality of CEP devices based on the dynamics of blood flow in the aortic arch.

2. The method of claim 1, wherein the vulnerability score is correlated with a risk that at least some of the plaque is detached during a surgical procedure applied to the aortic valve.

3. The method of claim 2, wherein the surgical procedure is a transcatheter aortic valve implantation (TAVI) procedure applied to the aortic valve.

4. The method of claim 3, wherein the vulnerability score is based on a type of implant to be used in the TAVI procedure.

5. The method of claim 2, wherein the vulnerability score is based on a total plaque volume and a spatial configuration of the plaque at or adjacent the aortic valve, and a fraction of lipid in the total plaque volume.

6. The method of claim 5, wherein the vulnerability score is further based on morphological factors associated with the plaque.

7. The method of claim 5, wherein the vulnerability score is determined by an artificial intelligence (AI) based model trained based on known outcomes of previous surgical interventions correlated with corresponding historical images of aortas, each of the historical images including a corresponding aortic valve.

8. The method of claim 5, wherein the determining that the CEP device should be deployed is based on the vulnerability score and not the branching angle, and wherein the CEP device to be deployed is selected based on the branching angle and not the vulnerability score.

9. The method of claim 1, wherein the one or more image is one or more computed tomography (CT) image, and the segmenting of the one or more image is implemented by an artificial intelligence (AI) based model to identify the aortic arch and a left subclavian artery (LSA).

10. The method of claim 9, further comprising:
identifying a branching angle between the LSA and the aortic arch, and
wherein the selecting of the CEP device is based on a fluid dynamics model of blood flow between the aortic arch and the LSA, the fluid dynamics model determining a likelihood that the plaque in the aortic arch will enter the LSA based on the identified branching angle.

11. The method of claim 10, wherein the fluid dynamics model is based on a size of the LSA, wherein the size of the LSA is determined from the one or more image or is independently known.

12. The method of claim 10,
wherein the plurality of branching blood vessels comprises a brachiocephalic artery, a left common carotid artery (CCA), and the LSA,
wherein a first CEP device of the plurality of CEP devices covers the brachiocephalic artery and the CCA but not the LSA, and
wherein a second CEP device of the plurality of CEP devices covers the brachiocephalic artery, the CCA, and the LSA.

13. The method of claim 1, wherein the branching angle is between a left subclavian artery (LSA) and the aortic arch, the method further comprising determining whether a size of the LSA is larger than a threshold size,
wherein a first CEP device of the plurality of CEP devices covers a brachiocephalic artery and a CCA but not the LSA, and wherein a second CEP device of the plurality of CEP devices covers the brachiocephalic artery, the CCA, and the LSA, and
wherein upon determining that the CEP device should be deployed and that either the LSA is larger than the threshold size or the branching angle is larger than a threshold angle, further determining that the second CEP device should be deployed.

14. A system for deploying a cerebral embolic protection (CEP) device, the system comprising:
a plurality of implantable CEP devices;
a memory having stored a plurality of instructions;
processor circuitry operatively coupled with the memory, the processor circuitry configured to execute the instructions to:
retrieve one or more image of at least part of an aorta, the one or more image including an aortic valve;
segment the one or more image to identify the aortic valve, an aortic arch, and a plurality of branching blood vessels downstream of the aortic valve;
identify plaque in a segment of the one or more image at or adjacent the aortic valve;
generate a vulnerability score associated with the plaque;
evaluate dynamics of blood flow in the aortic arch and at least one of the plurality of branching blood vessels;
determine that the CEP device should be deployed based on the vulnerability score;
select the CEP device from the plurality of implantable CEP devices based on at least one of (i) the dynamics of blood flow in the aortic arch or (ii) a branching angle identified between one of the plurality of branching blood vessels and the aortic arch.

15. The system of claim 14, wherein the surgical procedure is a Transcatheter aortic valve implantation (TAVI) procedure applied to the aortic valve.

16. The system of claim 14, wherein the vulnerability score is based on a total plaque volume and a spatial configuration of the plaque at or adjacent the aortic valve, and a fraction of lipid in the total plaque volume.

17. The system of claim 14, wherein the vulnerability score is determined by an artificial intelligence (AI) based model trained based on known outcomes of previous surgical interventions correlated with corresponding historical images of aortas, each of the historical images including a corresponding aortic valve.

18. The system of claim 14,
wherein the one or more image is one or more spectral computed tomography (CT) image, and the the processor circuitry is configured to execute the instructions to segment the one or more image by implementation of an artificial intelligence (AI) based model to identify the aortic arch and a left subclavian artery (LSA),
wherein the identified branching angle is between the LSA and the aortic arch, and
wherein the selection of the CEP device is based on a fluid dynamics model of blood flow between the aortic arch and the LSA, the fluid dynamics model configured to determine a likelihood that the plaque in the aortic arch will enter the LSA based on the identified branching angle.

19. The system of claim 14,
wherein the branching angle is between a left subclavian artery (LSA) and the aortic arch,
wherein the processor circuitry is configured to execute the instructions to determine
whether a size of the LSA is larger than a threshold size,
wherein a first CEP device of the plurality of implantable CEP devices covers the brachiocephalic artery and the CCA but not the LSA when deployed,
wherein a second CEP device of the plurality of implantable CEP devices covers the brachiocephalic artery, the CCA, and the LSA when deployed, and
wherein upon determination that the CEP device should be deployed and that either the LSA is larger than the threshold size or the branching angle is larger than a threshold angle, the processor circuitry is configured to execute the instructions to further determine that the second CEP device should be deployed.

20. The system of claim 14, further comprising a computed tomography imaging device, and wherein the processor circuitry is configured to execute the instructions to retrieve the one or more image from the computed tomography imaging device.
